(19) United States
(12) Patent Application Publication
(10) Pub. No.: US 2016/0121062 A1
(43) Pub. Date: May 5, 2016

(54) High Frequency Airway Oscillation For Internal Airway Vibration

(71) Applicant: UNIVERSITY OF FLORIDA RESEARCH FOUNDATION, INC., Gainesville, FL (US)

(72) Inventors: Paul DAVENPORT, Gainesville, FL (US); Satyanarayan HEGDE, Chicago, IL (US)

(21) Appl. No.: 14/992,359

(22) Filed: Jan. 11, 2016

Related U.S. Application Data
(63) Continuation-in-part of application No. PCT/US2014/048632, filed on Jul. 29, 2014.
(60) Provisional application No. 61/860,547, filed on Jul. 31, 2013.

(51) Int. Cl. A61M 16/00; A61H 23/02

(52) U.S. Cl. CPC A61M 16/0006; A61H 23/02

(57) ABSTRACT

The current invention pertains to methods of clearing mucus from airways of patients using devices for applying high frequency oscillations to the air passing through the airways of the patients. The devices can comprise a mouthpiece and a high frequency oscillator operably connected to the mouthpiece. The devices create turbulence throughout the airways of the patient from the mouth to the alveoli when the patient breathes through the mouthpiece, thereby clearing the mucus from the airways and helping the patient breathe easily. In certain embodiments, the device is a portable device. In further embodiments, the device is battery operated. In further embodiments, mucus cleared from the airways of patients and/or exhaled breath samples from the patients are collected for analysis and diagnosis during and/or following application of high frequency oscillations.

### CROSS-REFERENCE TO A RELATED APPLICATION

This application is a continuation-in-part of International Application PCT/US2014/048632, filed Jul. 29, 2014; which claims the benefit of U.S. provisional application Ser. No. 61/860,547, filed Jul. 31, 2013, all of which are incorporated herein by reference in their entirety.

### BACKGROUND OF INVENTION

Mucus is continually produced in the lungs and keeps the airways moist. Particles of dust, dirt or bacteria lodge in the mucus, which is cleared in the healthy lung and swallowed. This process happens all of the time and is the way that the lungs keep themselves clear and free of infection.

There are several respiratory tract infections or diseases that involve sputum/mucus formation that can block the airways of the patients. For example, cystic fibrosis is an inherited disease that damages vital organs, especially the lungs and pancreas, by clogging them with mucus. Cystic fibrosis (CF) patients suffer from the production of abnormal mucus that is excessively thick and sticky. As a result, the process of cleaning of the lungs is inefficient or absent leading to build-up of bacteria, dirt and mucus in the lungs. Infection as a result is more likely. Drugs exist which can ameliorate its effects, but physical management of the disease is nevertheless very important. There are many other lung diseases like CF that cause excessive mucus production (mucus hypersecretion) and will lead to similar problems seen in CF.

Treatments for excessive mucus in the airways of the patients, for example, CF patients, include mechanically breaking the mucus, for example, by chest physiotherapy, chest clapping technique, or chest percussion therapy. However, these methods have disadvantages, for examples, these therapies are not appropriate for patients who have just eaten or are vomiting, have acute asthma or tuberculosis, have brittle bones or broken ribs, are bleeding from the lungs or are coughing up blood, are experiencing intense pain, have increased pressure in the skull, have head or neck injuries, have collapsed lungs or a damaged chest wall, recently experienced a heart attack, have a pulmonary embolism or lung abscess, have an active hemorrhage, have injuries to the spine, have open wounds or burns, or have had recent surgery. Also, these treatment methods require a person to administer the therapy and the patient is awake or is awakened during the therapy, which is a significant problem, especially when the patients are children. Further, these treatments can only be administered in an intermittent manner and cannot provide a continuous relief from mucus problems in patients. Therefore, alternate methods and devices of clearing the mucus from the patients' airways in a continuous manner without involvement of a person administering the therapy and without disturbing the patients are desirable.

In addition to treatment, there is increased interest in providing a simple and efficient method for diagnosing respiratory infections that involve or develop as a result of sputum/mucus formation that blocks the airways of the patient. Such respiratory infections include influenza, parainfluenza, adenovirus, respiratory syncytial virus, human metapneumovirus, SARS, MERS, and Rhinovirus.

Chronic bronchitis in children often requires bronchoalveolar lavage (BAL) to identify the bacteria causing underlying infections. Patients with cystic fibrosis (CF) develop chronic bronchitis and require frequent BAL. However, BAL is an invasive test that requires sedation and passing an endoscope through the patients' windpipe. Moreover, microbial infections are diagnosed by culturing them in growth media, which is not a very sensitive method.

A good sputum sample would be equal to BAL. However, most young children and many CF patients cannot produce sputum despite having significant mucus accumulation in their lungs. There are no currently available systems for noninvasively inducing and collecting a valid sputum sample from CF and other patients suffering from respiratory infections. Therefore, an optimized non-invasive sputum collection system for diagnosis is desirable.

### BRIEF SUMMARY

Embodiments of the invention are directed to systems and methods for clearing mucus from airways of a patient using a means for generating and/or maintaining an oscillating airflow. In certain embodiments, a device is utilized that applies high frequency oscillations to the air passing through the airways of the patient, wherein the device comprises a mouthpiece and a high frequency oscillator operably connected to the mouthpiece. The device creates turbulence throughout the airways of the patient from the mouth to the alveoli when the patient breathes through the mouthpiece, thereby clearing the mucus from the airways and helping the patient breathe easily. In certain embodiments, the device is a portable device. In further embodiments, the device is battery operated.

In related embodiments, during and/or following application of an oscillating airflow to a patient, sputum and/or exhaled breath samples are obtained from the patient that are analyzed for substances associated with infection.

In certain embodiments, a method is provided for internal airway percussion (IAP) in which the transmission of acoustic sound waves into the lower respiratory tract is performed for effective vibration of the lung. Acoustic waves produced by IAP vibrate both the upper and lower respiratory tracts, thus increasing the release of aerosolized bioparticles (including microbes) into the exhaled breath.

### BRIEF DESCRIPTION OF DRAWINGS

FIG. 1 shows an embodiment of a high frequency airway oscillation system for clearing mucus in airways of a patient and/or obtaining a sputum sample or exhaled breath particles for analysis in accordance with the invention.

FIG. 2 shows another embodiment of a high frequency airway oscillation system for clearing mucus in airways of a patient and/or obtaining a sputum sample or exhaled breath particles for analysis in accordance with the invention.

FIG. 3 shows yet another embodiment of a high frequency airway oscillation system for clearing mucus in airways of a patient and/or obtaining a sputum sample or exhaled breath particles for analysis in accordance with the invention.

FIG. 4A shows an embodiment of a device for supplying oscillating airflow to a patient.

FIG. 4B is a picture of an embodiment of a device for supplying oscillating airflow to a patient.

FIGS. 5A-5F are illustrations of observed respiratory parameters in control and IAP groups in humans. FIG. 5A is a graphical illustration of the pattern of changes in ETCO₂ with time. FIG. 5B is a graphical illustration of mean ETCO₂. FIG. 5C is a graphical illustration of the pattern of changes in heart rate with time. FIG. 5D is a graphical illustration of mean heart rate. FIG. 5E is a graphical illustration of the pattern of changes in respiratory frequency with time. FIG. 5F is a graphical illustration of mean respiratory frequency. The * indicates a significant difference, p<0.05.

FIG. 6 is a graphical illustration of exhaled protein concentration in control and IAP trials in humans in 5 Hz and 15 Hz groups. The ** indicates a significant difference, p<0.01.

FIGS. 7A-7D are illustrations of observed respiratory parameters in control and IAP groups in dogs. FIG. 7A is a graphical illustration of the pattern of changes in ETCO₂ with time. FIG. 7B is a graphical illustration of mean ETCO₂. FIG. 7C is a graphical illustration of the pattern of changes in heart rate with time. FIG. 7D is a graphical illustration of mean heart rate.

FIG. 8 is a graphical illustration of exhaled protein concentration in control and IAP trials in dogs. The ** indicates a significant difference, p<0.01.

FIGS. 9A-9D are graphical illustrations of respiratory perception in humans. FIG. 9A is a graphical illustration of air hunger. FIG. 9B is a graphical illustration of the effort of breathing. FIG. 9C is a graphical illustration of the effect of unpleasantness. FIG. 9D is a graphical illustration of the effect of suffocation. The “C” stands for Control trial; H stands for IAP trial.

FIG. 10 is a schematic of the experimental set-up described in Example 4 that uses an embodiment of a device of the subject application.

FIG. 11 is a table listing pertinent characteristics of subjects described in Example 4.

FIG. 12 is a table summarizing statistical p-values of total mass and representative sizes of bioaerosol for nine examined operation conditions analyzed by Wilcoxon signed-rank test as described in Example 4.

FIG. 13 is a table summarizing average particle mode size (in μM) of all combinations after ten minute sampling as described in Example 4.

FIGS. 14A-14D show the effect of IAP device application on total collected mass of particles sampled from the exhaled breath (EB) at two tested conditions: FIG. 14A shows total mass of particles collected from the EB of all human subjects by Combination 3; FIG. 14B shows total mass of particles collected from the EB of all human subjects by Combination 4; FIG. 14C shows total mass ratio for Combination 3; and FIG. 14D shows total mass ratio for Combination 4.

FIGS. 15A-15D show the effect of IAP device application on mode and median sizes of the particles sampled from the EB of Combination 3 and Combination 4 at two tested conditions (error bars indicate 95% confidence): (FIG. 15A) particle mode size ratio of Combination 3 with acoustic waves at 15 Hz and pressure intensity of 3.0 cm of H₂O; (FIG. 15B) particle mode size ratio of Combination 4 with acoustic waves at 30 Hz and pressure intensity of 0.75 cm of H₂O; (FIG. 15C) particle median size ratio of Combination 3 with acoustic waves at 15 Hz and pressure intensity of 3.0 cm of H₂O; (FIG. 15D) particle median size ratio of Combination 4 with acoustic waves at 30 Hz and pressure intensity of 0.75 cm of H₂O.

FIGS. 16A-16E show average particle size distribution of the collected particles from the EB of the subjects at different sampling times for Combination 3 at: (FIG. 16A) 2^{nd} min, (FIG. 16B) 4^{th} min, (FIG. 16C) 6^{th} min, (FIG. 16D) 8^{th} min, and (FIG. 16E) 10^{th} min.

### DETAILED DISCLOSURE

The term “about” is used to describe certain aspects of the current invention, for example, frequency of oscillations or duration of treatment administration. It should be understood that mathematical accuracy is not required with respect to these aspects for the invention to operate and the corresponding parameters can be altered by ±10% without affecting the operability of the invention. For example, oscillation frequency of about 300 Hz corresponds to oscillation frequency of anywhere between 270 Hz to 330 Hz.

For the purposes of this invention, clearing the mucus from airways of a patient means releasing the mucus from the internal walls of the airways and moving the mucus towards the mouth for expulsion.

A mouthpiece refers to a receptacle designed to be put in or against the mouth of a patient through which the patient can breathe.

For the purposes of this invention, “operably connected” means connected in a manner that allows flow of air to and from the two connected portions or parts. E.g., a high frequency oscillator operably connected to a mouthpiece means that air can flow to and from the oscillator to the mouthpiece.

As defined herein, a patient is a mammal to which the means for maintaining and generating oscillating airflow is applied. Mammalian species that benefit from the disclosed systems and methods include, but are not limited to, humans, apes, chimpanzees, orangutans, monkeys, and domesticated animals such as dogs, cats, mice, rats, guinea pigs, hamsters, horses, cows, and anesthetized wild animals, including aquatic mammals.

An adult for the purposes of the invention is a patient over eighteen (18) years of age; whereas, a pediatric patient is a patient under eighteen (18) years of age. Pediatric patients include infants, children and adolescents.

A “sputum sample” refers to mucus that is cleared from the patient in accordance with the subject invention. According to the invention, a sputum sample or exhaled breath particles are used for microbiological investigations of respiratory infections and cytological investigation of respiratory systems. The sputum sample preferably contains very little saliva.

The present invention provides a method for clearing mucus from airways of a patient using a means for generating and/or maintaining an oscillating airflow. An embodiment of the invention involves the step of transmitting acoustic sound waves into the respiratory tract of a patient to vibrate the upper and/or lower respiratory tracts to induce release of bioparticles into exhaled breath. In certain embodiments, a device is provided for applying a high frequency oscillation to the air passing through the airways of the patient, wherein the device comprises a mouthpiece and a high frequency oscillator operably connected to the mouthpiece. The high frequency oscillator creates turbulence throughout the airways of the patient from the mouth to the alveoli when the patient breathes through the mouthpiece thereby releasing the mucus attached to the inside of the patient's airways and moves the mucus towards the mouth for expulsion. The patient can be a patient diagnosed with cystic fibrosis. The patient can be an adult or a pediatric patient.

The current invention is different from the existing technologies because the existing technologies vibrate the outside of the chest to vibrate the airway. The current invention is also an advancement over the existing technology, for example, flutter valve and lung flute, which require active expiratory breathing tasks to produce an internal airway vibration. The flutter valves and lung flute vibration magnitude and frequency is a function of the expiratory breathing force and cannot be regulated by the clinician and only provides a vibration during the exhalation phase of the breath; wherein in the devices of the current invention, the frequency and amplitude are set and controlled by the clinician allowing specific active internal airway percussion modifications for individual patients.

According to the subject invention, the means for generating and/or maintaining an oscillating airflow include those devices that induce intra-thoracic oscillations. Intra-thoracic oscillations are generated orally, nasally and/or endotracheally and are created using variable frequency and amplitude pressure or airflow pump producing air force waves within the airways generating controlled oscillating positive pressure. When oscillation frequency approximates the resonance frequency of the pulmonary system, endobronchial pressure oscillations are amplified and result in vibrations of the airways and lungs. The intermittent increases in endobronchial pressure reduce the collapsibility of the airways during exhalation, thereby mobilizing the release of increased concentration of materials (such as biomarkers and/or organisms) in exhaled breath as compared with exhaled breath condensates (EBC) released in exhaled breath sampled without oscillating airflow. Conventional EBC sampling possesses variable and extremely high dilution factor that is often beyond the limits of target detection. According to the subject invention, external means for generating and/or maintaining oscillating airflow does not include humming. Humming is normally used as a way to vibrate air and increase movement of molecules out of the nasal airways. In contrast, the external oscillating airflow means of the invention is a mechanical device that applies oscillating air force waves beyond the nasal passages and into the lungs and airways. Methods and devices currently available for inducing intra-thoracic oscillations and for generating and/or maintaining oscillating airflow to a patient include, but are not limited to: flutter devices (devices that contain ball bearings that repeatedly interrupt the outward flow of air from a patient); acapella devices (flow operated oscillatory positive expiratory pressure (PEP) device that uses a counterweighted plug and magnet to generate oscillatory forces); cornet devices (tubes that house inner tubes where the rotation of the inner tube reflects resistance generated in airflow—as the patient exhales through the outer tube, the inner tube unfurls generating a rhythmic bending and unbending of the inner tube throughout the expiration phase); intrapulmonary percussive ventilation devices (also known as IPV devices that provide continuous oscillation to the airways via the mouth, endotracheal tube, or nose); and other devices that provide forced oscillation or impulse oscillometry. In one embodiment, an IPV device that applies vibratory air pressure waves superimposed on the breath airflow is used to generate and/or maintain oscillating airflow to a patient. Examples of such IPV devices are described in U.S. Pat. Nos. 6,595,213 and 6,695,978, both of which are incorporated herein in their entirety. In another embodiment, an oscillating device such as that disclosed in U.S. Pat. No. 4,333,476 is used to generate and/or maintain oscillating airflow to the patient.

In one embodiment, the external means for generating and/or maintaining oscillating airflow to the patient is a device that uses the same principle as a loud speaker. Preferably, the external means for generating and/or maintaining oscillating airflow comprises an electroacoustic transducer (speaker) that produces sound in response to an electrical audio signal. Sound, as defined herein, is a mechanical wave that is an oscillation of pressure transmitted through some medium (like air or water). In a particular embodiment, a speaker is used to transmit high frequency square form audio waves to the airways of the patient. In another embodiment, a speaker is used to transmit low frequency square form audio waves to the airways of the patient. In yet another embodiment, the external means for generating and/or maintaining oscillating airflow to the patient is a device that uses the same principle as a piston pump.

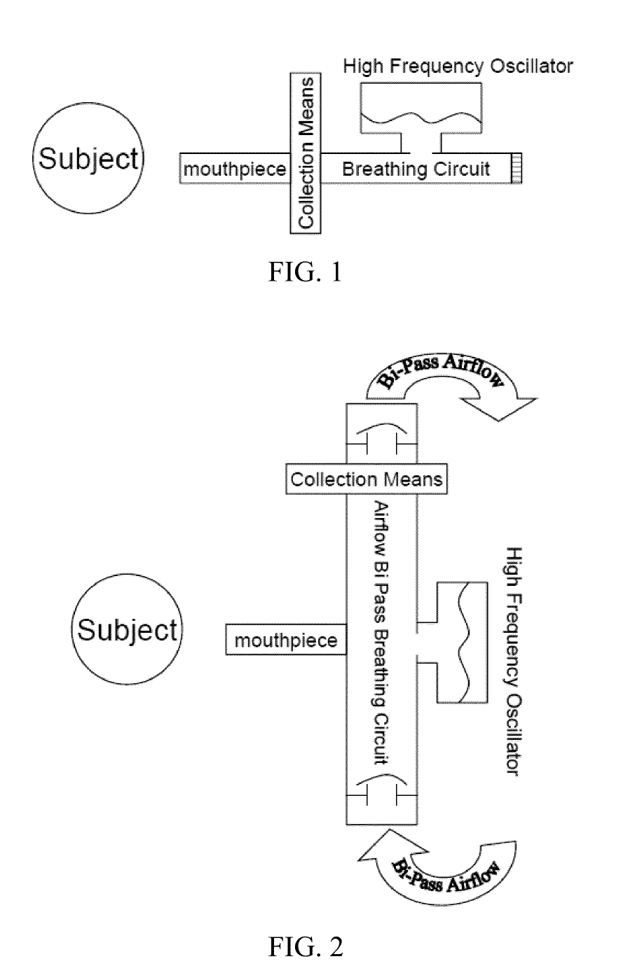

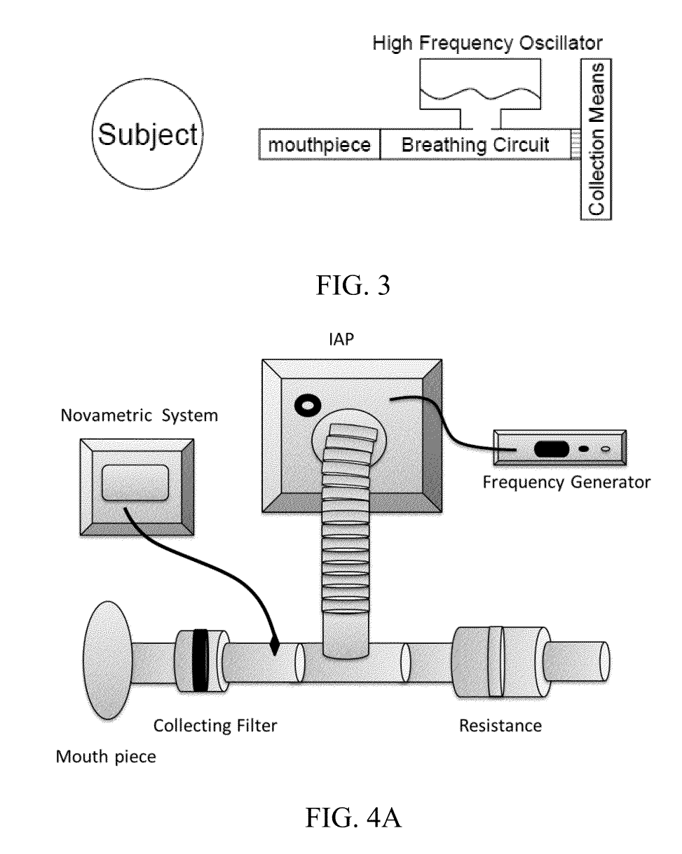

One oscillating system for use in accordance with the invention includes the Jaeger Master Screen Impulse Oscillator System (Viasys, Inc.). This device uses a fixed frequency and amplitude oscillation of a speaker attached as a side arm to a breathing circuit. An oscillating electrical current is applied to a speaker (or piston pump) to generate an inflating and deflating pressure force. This force is applied as a side-arm on a breathing circuit (or tube) through which the patient is inhaling and/or exhaling (see FIGS. 1-3). As illustrated in FIGS. 1-3, the pressure force is superimposed on the airflow through the tube (or circuit) that is the breathing air produced by the patient.

In certain embodiments, the device for inducing intra-thoracic oscillations comprises a mouthpiece and a high frequency oscillator operably connected to the mouthpiece, wherein the high frequency oscillator creates turbulence throughout the airway from the mouth to the alveoli when the subject breathes through the mouthpiece. The high frequency oscillator can be connected to the mouthpiece via a side port.

According to the invention, external oscillating airflow is applied as a superimposed oscillating pressure-flow force to normal breathing for single or multiple breaths. The oscillator is applied with large breaths and/or forced breaths. As illustrated in FIGS. 1-3 the outflow of the patient's air passes over the collection means. The oscillation can be present during both inhalation and exhalation, although oscillation can also be applied only during inhalation or only during exhalation. The oscillation can also be applied during breathing behaviors such as cough, large breaths and forced exhalations.

In certain embodiments, the device used for inducing intra-thoracic oscillations is a battery powered device. In further embodiments, the device is a portable device and can be used by a patient on an outpatient basis (e.g., outside of a clinician's office) to clear the patient's airways of mucus in a continuous manner.

The methods and devices described above for inducing intra-thoracic oscillations and generating and/or maintaining oscillating airflow to a patient are preferably applied as a superimposed oscillating pressure-flow force to normal breathing for single or multiple breaths. Such methods and devices can also be applied with large breaths and forced breaths. In certain embodiments, oscillation is applied to a patient only during inhalation or only during exhalation. In such embodiments, one configuration of the breathing circuit is to use a non-breathing valve to separate the inhalation and exhalation tubes. In other embodiments, oscillation is applied to a patient during both inhalation and exhalation. Following initial application, the frequency and amplitude of the pressure-flow oscillation applied to a patient is adjusted to optimize the mucus to be removed from the patient.

According to the invention, oscillating frequency can range from about 0.5 Hz to about 1,000 Hz. In one embodiment, the oscillating frequency is in the range of about 1 Hz to about 500 Hz, about 5 Hz to about 100 Hz, to about 5 Hz to about 50 Hz, and about 10 Hz to about 300 Hz. In yet another embodiment, the oscillating frequency is in the range of about 500 Hz to about 1,000 Hz, about 700 Hz to about 1,000 Hz, and about 800 Hz to about 1,000 Hz. In further embodiments, acoustic sound waves at about 5 Hz, 10 Hz, 15 Hz, 20 Hz, 25 Hz, 30 Hz, 35 Hz, 40 Hz, 45 Hz, 50 Hz, 55 Hz, 60 Hz, 65 Hz, 70 Hz, 75 Hz, 80 Hz, 85 Hz, 90 Hz, 95 Hz, or 100 Hz are administered to a patient at varying oscillating amplitudes (intensities).

In certain embodiments, the oscillating amplitude is in the range of 0.5-15 cm H₂O pressure. In certain embodiments, the oscillating amplitude is at about 0.75 cm H₂O, 1.5 cm H₂O, and 3 cm H₂O. In other embodiments, the oscillating volume ranges between 1-20% of total lung capacity. The amount of time oscillating forces are administered to a patient will be determined by the amount of sample to be collected.

In one embodiment, the oscillating forces are administered to a patient from about 1 minute to about 3 hours, from about 1 minute to about 1 hour, from about 1 minute to about 30 minutes. In another embodiment, the oscillating forces are administered to a patient from about 5 minutes to about 20 minutes. Those skilled in the art can readily adjust oscillating frequency, amplitude, volume, and amount of administration time in relation to the patient, lung capacity, and airway diameters.

In one embodiment of the subject invention, the frequency and amplitude of the oscillating pressure can be constant based on the optimum frequency for clearing mucus from a patient's airways. In a related embodiment, the frequency of oscillating pressure is varied to the optimum frequency for moving mucus from a patient's airways. In this embodiment, the frequency and amplitude is adjusted to the airway as a function of the trachea diameter and total lung capacity, which are important for the size of the patient (such as adult versus pediatric human sizes and other animal species).

According to the subject invention, following application of oscillating pressure-flow force to a patient, a sputum sample can be collected from the patient. Methods for obtaining a sputum sample from a patient comprises the steps of: supplying an external means for generating and/or maintaining an oscillating airflow to a subject; collecting at least one sputum sample following application of the oscillating airflow; and assessing the biomarkers and/or organisms present in the sputum breath sample. For example, an adequate sputum sample may be obtained when expulsed mucus from the patient is directed to a collection means (as depicted in FIGS. 1-3). In yet another related embodiment, following sputum sample analysis, the subject is diagnosed with regard to health status, including diagnosis of any diseases and/or conditions associated with biomarkers and/or organisms present in the sputum sample.

The systems of the present invention for noninvasively obtaining a sputum sample or exhaled breath particles include the following parts: 1) an oscillating pressure means to be applied to the subject's airflow during inhalation and/or exhalation; and 2) a sputum collection means. Certain embodiments further comprise a sensor having the ability to detect and/or quantify biomarkers and/or organisms present in exhaled breath. In related embodiments, the sensor is coupled to a processor, which can store, track trend, and interpret the sensor signals to provide useful information regarding biomarker and/or organism amount or concentration for display to the user.

A collection means is any suitable containment method or device for containing an exhaled breath and/or sputum sample taken from a patient. The collection means can be a receptacle for collecting exhaled breath and/or mucus expulsed following application of oscillating pressure-flow force to a patient. Such receptacles include, but are not limited to, tubes, vials, strips, capillary collection devices, cannulas, and miniaturized etched, ablated or molded flow paths. The collection means can be a material, such as an absorbent material, used to collect gases and/or liquids. Examples of absorbent material for use in accordance with the invention include, but are not limited to, sponge-like materials, hydrophilic polymers, activated carbon, silica gel, activated alumina, molecular sieve carbon, molecular sieve zeolites, silicalite, AIPO₄ alumina, polystyrene, TENAX series, CARBOTRAP series, CARBOPACK series, CARBOXEN series, CARBOSEIVE series, PROAPAK series, SPHEROCARB series, DOW XUS series, and combinations thereof. In one embodiment, the collection means is a sterile TEDLAR® bag for storing exhaled breath containing aerosolized bioparticles. In certain embodiments, the collection means can include both a receptacle and material described above. Those skilled in the art will know of other suitable receptacles and absorbent materials for use in accordance with the invention.

In certain embodiments, collected sputum sample or exhaled breath particles are subjected to sensors for detection and/or quantification of biomarkers and/or organisms present in the sample. Sensors of the subject invention can include commercial devices commonly known as “artificial” or “electronic” noses or tongues. Other sensors for use in accordance with the subject invention include, but are not limited to, metal-insulator-metal ensemble (MIME) sensors, cross-reactive optical microsensor arrays, fluorescent polymer films, surface enhanced raman spectroscopy (SERS), diode lasers, selected ion flow tubes, metal oxide sensors (MOS), bulk acoustic wave (BAW) sensors, calorimetric tubes, infrared spectroscopy, semiconductive gas sensor technology; mass spectrometers, fluorescent spectrophotometers, conductive polymer gas sensor technology; aptamer sensor technology; amplifying fluorescent polymer (AFP) sensor technology; microcantilever technology; molecularly polymeric film technology; surface resonance arrays; microgravimetric sensors; thickness sheer mode sensors; surface acoustic wave gas sensor technology; radio frequency phase shift reagent-free and other similar micromechanical sensors.

Specific biomarkers that are collected and measured in sputum for use in diagnosis of disease or condition in accordance with the subject invention include, but are not limited to, alveolar macrophages, lung eosinophils, bacteria, H₂O₂, adenosine, nitrate (NO₃⁻) and nitrite (No₂⁻), nitrotyrosine, nitrosothiols (RS—NOs), arachidonic acid metabolites (such as prostaglandins and thromboxanes), leukotrienes (such as leukotriene (LT)C₄, LTD₄, LT₄)), 8-isoprostanes, aldehydes (such as malondialdehyde, 4-hydroxyhexanal, 4-hydroxynonenal, hexanal, heptanal, and nonanal), ammonia (NH₃ and NH₄), cytokines, p53 mutation, DNA hepatocyte growth factor, vitronectin, endothelinl, chemotactic activity, DNA fragments, RNA fragments, proteins, angiogenic markers (such as vascular endothelial growth factor, basic fibroblast growth factor and angiotension), and inflammatory markers (such as tumor necrosis factor-α, interleukin 6).

Specific organisms that are collected and measured in sputum for use in diagnosis of disease or condition in accordance with the subject invention include, but are not limited to, different species of bacteria, such as Pseudomonas, Mycobacteria, Staphylococcus, MRSA, Klebsiella, Pneumococci, Acinetobacter, Burkholderia, Chlamydia, Hemophilus, Moraxella, Serratia, Enterobacter, Stenotrophomonas, and Citrobacter; fungi, such as Candida, Aspergillus, Histoplasma, Coccidiomycosis, Blastomycosis, Pneumocystis jiroveci, Cryptococcus, and Sporotrichosis; and viruses, such as influenza, parainfluenza, adenovirus, respiratory syncytial virus, human metapneumovirus, SARS, MFRS, and rhinovirus.

Diseases and conditions that can be diagnosed in accordance with the subject invention include, but are not limited to, inflammatory conditions, airway infections, common-cold, tumors, drug-related effects, and anatomical abnormalities. Specific diseases or conditions include, but are not limited to; asthma, CF, tuberculosis, chronic obstructive pulmonary diseases, bronchiectasis and acute respiratory distress syndrome, acute hypoxaemic respiratory failure, reperfusion injury, allergic rhinitis, system sclerosis, respiratory tract infection, bacterial pneumonia, interstitial lung disease, pulmonary sarcoidosis, obstructive sleep apnea, ozone-inhalation, acute lung injury, and respiratory cancers including lung cancer. All of these diseases or conditions can be diagnosed by analyzing samples collected in accordance with the subject invention using morphologic, immunochemical, fluorescence, molecular, or genetic techniques.

In certain embodiments, an exhaled breath sample is obtained from the patient. The concentration of biomarkers and/or organisms in oral exhaled breath is greatly increased by the presence of an oscillating airflow provided to patients. Moreover, the invention increases the amount of substances exhaled that are normally present on the lining of the airways in the lung (such as cells and bacteria) and not normally exhaled in readily detectable concentrations.

According to the subject invention, methods for obtaining an exhaled breath sample from a patient comprise the steps of: supplying an external means for generating and/or maintaining an oscillating airflow to a subject; collecting at least one exhaled breath sample following application of the oscillating airflow; and assessing the exhaled breath sample to identify and/or quantify biomarkers and/or organisms present in the sample. For example, the exhaled breath sample is analyzed for biomarkers and/or organisms, which can include identification and/or measurement of concentration of specific biomarkers and/or organisms present in the sample. In yet another related embodiment, following exhaled breath sample analysis, the subject is diagnosed with regard to health status, including diagnosis of any diseases and/or conditions associated with biomarkers and/or organisms present in the exhaled breath sample.

The following examples illustrate materials and procedures for making and practicing the invention. These examples should not be construed as limiting. All percentages are by weight and all solvent mixture proportions are by volume unless otherwise noted. It will be apparent to those skilled in the art that the examples involve use of materials and reagents that are commercially available from known sources, e.g., chemical supply houses, so no details are given respecting them.

### Example 1

Dogs with veterinary clinical diagnosis of lung disease and bacterial pneumonia will be anesthetized. Their exhaled air will be sampled with three protocols in this example: 1) the dogs will be anesthetized and mucosal surface nasal and oral samples will be collected directly by means of sterile probe, 2) the dogs will then quietly breathe through a collection filter for 10-20 minutes, 3) a high frequency air pressure oscillation (HFO) experimental protocol will then be presented as the dog breathes through a collection filter with the HFO device superimposing the air pressure oscillation on the normal tidal breath to vibrate the lung airway.

The dogs will be prepared for an anesthetized diagnostic procedure. An intravenous catheter will be placed and anesthesia induced with a slow intravenous bolus (over at least 1 minute to prevent apnea) of propofol (4-8 mg/kg). This will be followed by an infusion of propofol (0.1-0.4 mg/kg/min), with the rate adjusted to maintain an appropriate level of anesthesia. The animals will be intubated. When a sufficient plane of anesthesia has been established, a sterile probe (swab) will be used to collect nasal and oral mucosal surface samples directly.

Following sampling of nasal and oral mucosal surface, the animals will have a non-rebreathing valve with an expiratory filter attached to the endotracheal tube. The dog will quietly breathe through the non-rebreathing valve, exhaling into a collection filter for 10-20 minutes.

Then, the HFO breathing device will be attached to the center chamber of the non-rebreathing valve and a new collection filter put into place. Air pressure will be oscillated at 10-300 Hz. The animal will breathe spontaneously with the HFO superimposed on the normal tidal volume. The animal will exhale through the collection filter with the HFO vibrating the lung airway for 5-20 minutes.

Following the experiment, all of the swabs and breathing filters collected in the experiment will be stripped for 10 minute in 10 ml phosphate buffer saline at room temperature, and the stripping solution will then be centrifuged at 8500 g for 10 minutes at room temperature. The supernatant will be decanted and stored for volatile organic compounds using HPLC and mass spectrometry. 100 μl phosphate buffer saline will be added to the precipitate pellets. The pellets will be analyzed via real-time PCR, microscopy, colony-forming assay and proteomic assays.

### Example 2

Below is a brief description of an example of a method of clearing airways of a patient by using the methods and devices of the current invention.

The patient breathes through a mouthpiece and connecting breathing circuit. The high frequency oscillator applies vibratory air pressure waves superimposed on the normal breath airflow. The high frequency oscillation (HFO) increases the kinetic motion of the gas molecules and creates turbulence throughout the airway from the mouth to the alveoli (air sacs). The turbulence vibrates the airways and lungs. The pressure vibrations shake the airways walls internally to facilitate the movement of mucus that lines the internal walls of the airways and move the mucus towards the mouth for expulsion. This is particularly important for cystic fibrosis patients that require airway vibration to assist the movement of their airway mucus towards the mouth.

The HFO acts throughout the breathing cycle, i.e. during both inhalation and exhalation. The HFO does not require any active breathing task for the patient, only regular breathing through a mouthpiece or facemask and breathing circuit with the HFO device generating pressure waves superimposed on breathing. This makes the device portable, easy to use and applicable to all age groups from infants to the elderly.

### Example 3

### Materials and Methods

### Conscious Human Study

### Participants

The human study was approved by the Institutional Review Board of the University of Florida. Seventeen healthy adults with no history of pulmonary or neurological disease participated in the study after providing informed written consent.

### Internal Airway Percussion (IAP) Device

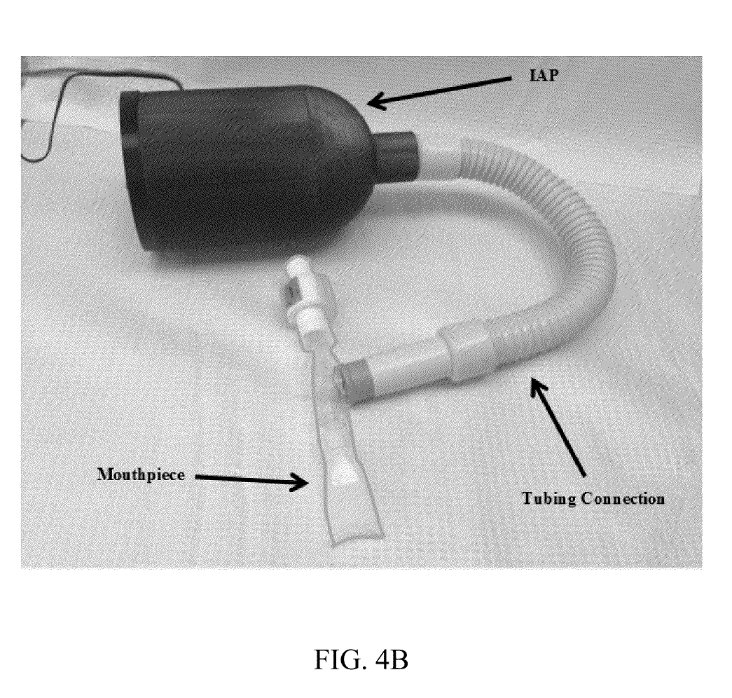

The IAP device was made from a speaker connected to a frequency generator and amplifier. The frequency generator allowed adjustment of the frequency of the percussion waves and the amplifier controlled the magnitude of the percussion pressure. The pressure of IAP square-wave was fixed at 1.29±0.10 cmH₂O. The IAP device was attached to a breathing circuit with a heat and moisture exchanger (Smiths Medical ASD, Keene, N.H.) using a plastic tube. The condensation foam in the heat and moisture exchanger was used as a filter to capture exhaled protein. A separate sterilized heat and moisture exchanger was connected to the breathing circuit for each breathing trial. A resistance (5 cmH₂O/L/sec) approximately equal to normal pulmonary resistance was placed at the end of a breathing circuit to promote transfer of the IAP pressure waveform into the airways. The experimental set up is shown in FIGS. 4A and 4B.

### Procedure

Participants, while sitting on a comfortable chair, were asked to breathe through a mouthpiece connected to the IAP breathing circuit with a collecting filter. The control group breathed through the mouthpiece with the IAP off for 20 minutes. The filter was removed from the circuit and placed in a separate storage bag. Subjects then were allowed at least a 10 minute break. In the IAP trial, a new filter was inserted into the breathing circuit and the subject again respired through the mouthpiece for 20 minutes with the IAP device activated. The trial order was not randomized because of the potential for the IAP to decrease the normal concentration of proteins in the respiratory tract. Use of IAP prior to non-IAP breathing could result in an IAP trial dependent decreased exhaled protein concentration in the control condition. Thus, the control trial always preceded the IAP trial.

A subgroup of subjects (n=5) were asked to estimate the magnitude of their sense of breathing effort, sense of suffocation, sense of air hunger and sense of unpleasantness using modified Borg scales from 0 (=no sensation) to 10 (=maximum) at the beginning of each trial (0 minute), 1 minute after a trial began (1 minute) and immediately after each trail was completed (20 minute).

### Respiratory Parameters

End-tidal CO₂ (ETCO₂), heart rate, respiratory frequency and IAP pressure were recorded during the entire experiment. The signal from monitor was led into a signal processing system (PowerLab, ADI Instruments, Castle Hill, Australia) and a desktop computer for continuous signal recording and analyzed using the LabChart 7 software.

### Protein Quantitation Analysis

Collecting filters were stored separately in a sterilized storage bag at 4° C. for less than 2 hours for the analysis of protein concentration. The foam was removed from the filter and placed into a 50 ml conical tube with 10 ml distilled water for 1 hr at room temperature. 100 μl of the solution was used and analyzed by NanoOrange® Protein Quantitation kit (Invitrogen, Carlsbad, Calif.) for the protein quantitation analysis.

### Statistical Analysis

The ETCO₂, heart rate and respiratory frequency with time were analyzed using two-way repeated measures ANOVAs with factors trial (control and IAP) and time (0 to 20 minute). Mean ETCO₂, mean heart rate, mean respiratory frequency and protein concentration were analyzed using one way repeated measures ANOVA. The ratings of breathing effort, suffocation, air hunger and unpleasantness were analyzed with one way repeated measures ANOVA. The significance criterion for all analyses was set at p<0.05.

### Anesthetized Dog Study

The study was approved by the University of Florida's IACUC. Seven dogs that were admitted to the Veterinary Hospital at the University of Florida for a routine dental cleaning were studied. The patient's medical care was under the supervision of the Veterinary Hospital at the University of Florida. Consent was obtained from clients prior to the IAP procedure. The dogs were anesthetized and endotracheal intubation performed. Prior to the dental clinical procedure, the IAP breathing circuit and device were connected between the endotracheal tube and an anesthesia machine. The control trial was breathing with IAP off for 10 minutes. Then the IAP breathing circuit was removed between the endotracheal tube and anesthesia machine. The IAP filter was removed from the circuit and placed in a separate sterilized storage bag. The animals were allowed at least a 5 minute rest period. Then the IAP trial was initiated. A new filter was inserted into the breathing circuit and the animal again respired through the filter containing breathing circuit for 10 minutes with the IAP device activated. ETCO2 and heart rate were recorded from the monitor every 30 seconds. The IAP breathing circuit was removed and the clinical procedure performed.

### Statistical Analysis

The ETCO2 and heart rate were analyzed using two-way repeated measures ANOVAs with factors trial (control and IAP) and time (0 to 10 minutes). Mean ETCO₂, mean heart rate and protein concentration were analyzed using one way repeated measures ANOVA. The significance criterion for all analyses was set at p<0.05.

### Results

### Respiratory Physiological Parameters

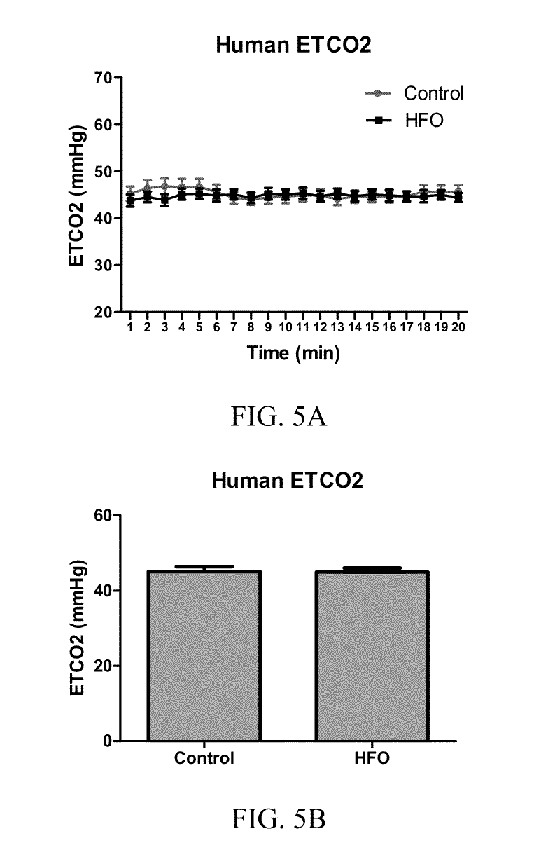

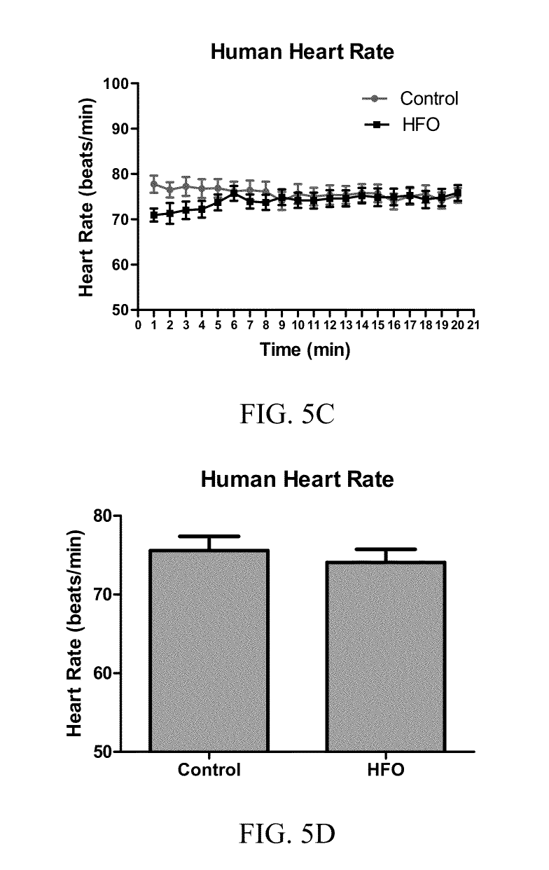

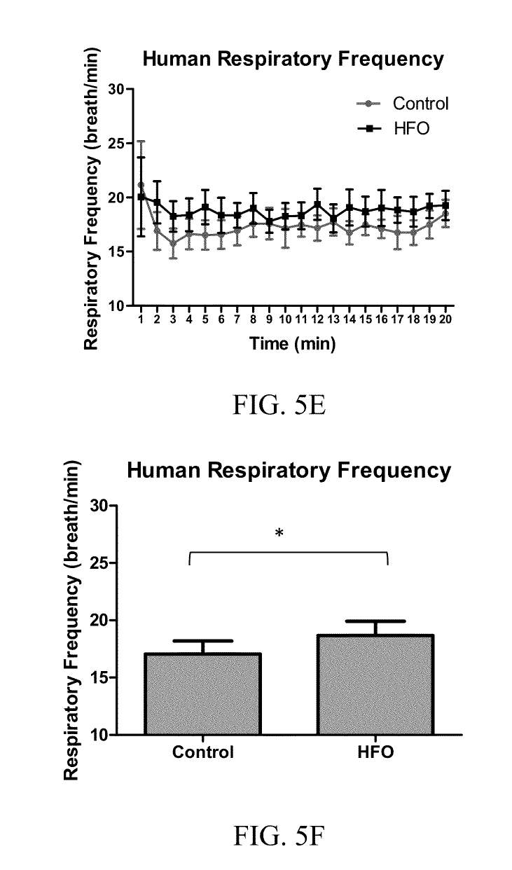

In the conscious human study, the results showed no significant differences in the ETCO₂ and heart rate with time between control and IAP trials (FIGS. 5A and 5C). In addition, there were no significant differences in mean ETCO₂ and mean heart rate between the two trials (FIGS. 5B and 5D). The IAP device significantly increased breathing frequency (p<0.05) compared to the control trial (FIGS. 5E and 5F).

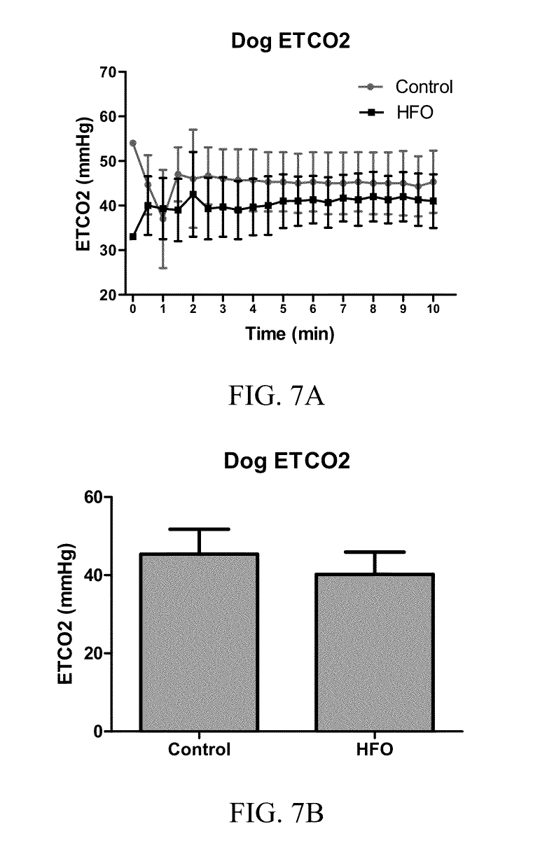

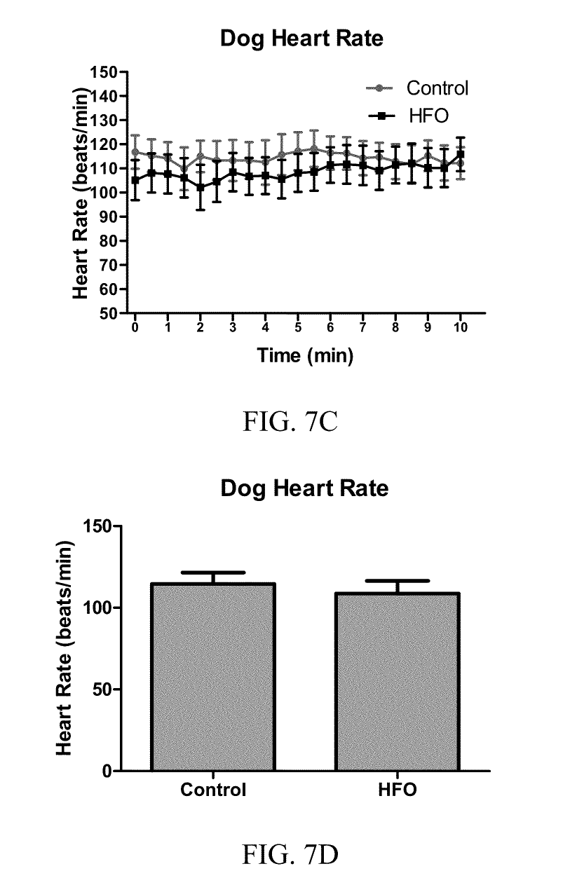

In anesthetized dogs, there were no significant differences in the ETCO₂, heart rate, mean ETCO₂ and mean heart rate (FIG. 7).

### Exhaled Protein Concentration

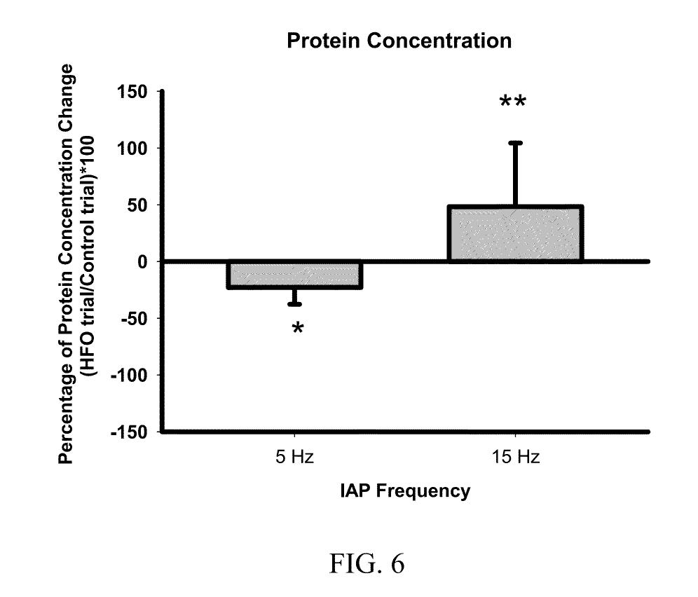

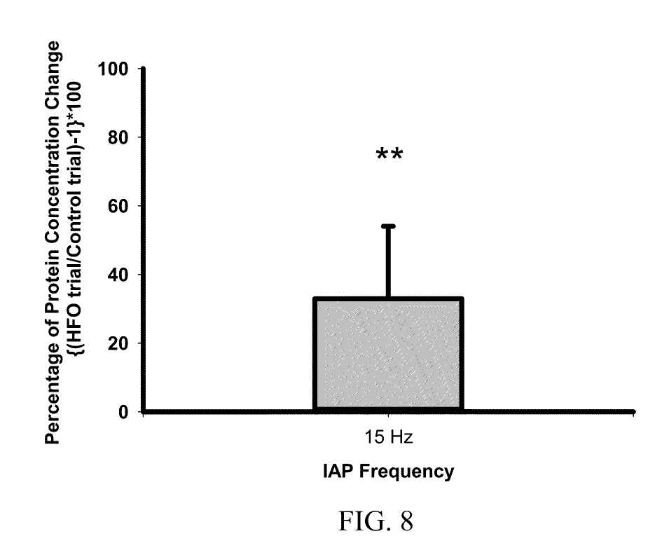

In conscious humans, square-wave IAP at 5 Hz significantly (p<0.01) decreased the protein concentration by 23% in the exhaled air filters compared to the control trial (FIG. 6). In contrast, square-wave IAP at 15 Hz significantly (p<0.01) increased the protein concentration in the exhaled air filters by 48% compared to control trial (FIG. 6). In anesthetized dogs, square-wave IAP at 15 Hz significantly (p<0.01) increased the protein concentration by 32% in exhaled air filters compared to the control trial (FIG. 8).

### Respiratory Perception

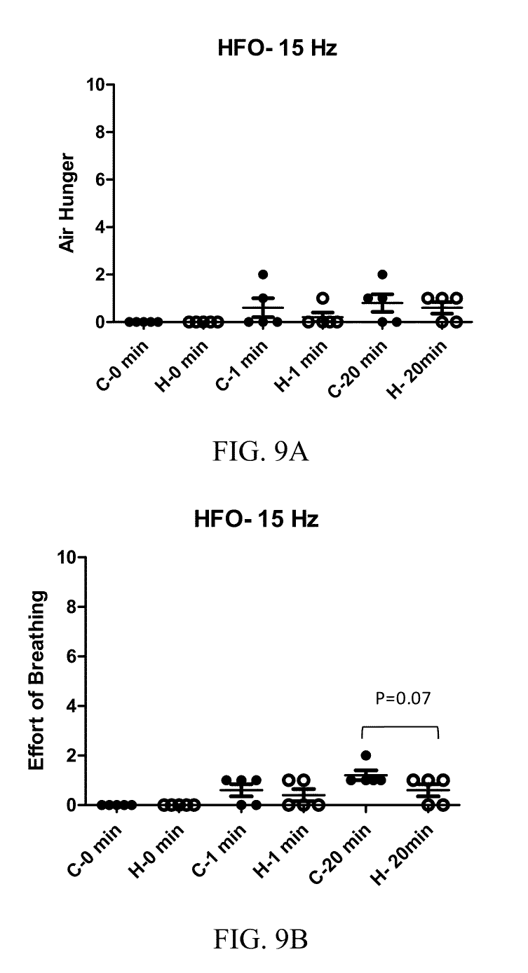

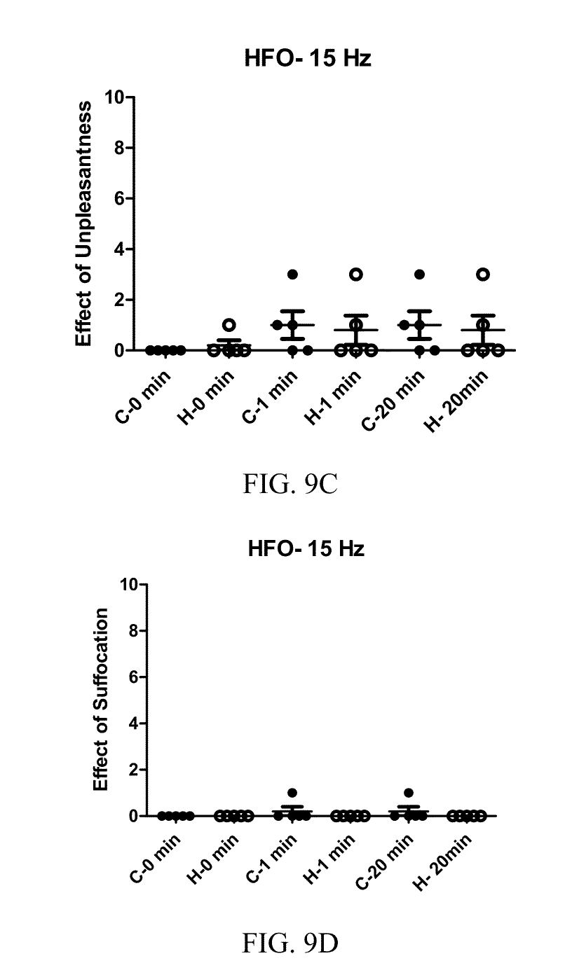

FIG. 9 shows that the IAP trial did not cause a change in the estimated magnitude of sensation of air hunger, unpleasantness or suffocation compared to control trial in conscious human. However, there was a trend for the magnitude estimation of breathing effort at 20 minutes to decrease with 15 Hz IAP (p=0.07).

In both conscious human participants and anesthetized dogs, the IAP device increased the concentration of protein in exhaled with 15 Hz, square-wave air percussion. This effect is frequency dependent because 5 Hz IAP did not increase but decreased the protein concentration in the exhaled air compared to control trials in humans. These results demonstrate that 15 Hz IAP has a greater effect on washing out the substances from respiratory tracts including mouth, tracheobronchial system and alveoli than control breathing and 5 Hz IAP. In the subject study, the control trial was performed prior to the IAP trail to ensure the changes in concentration of exhaled protein resulted from the effect of IAP not from the sequence of collection.

In the conscious human study, IAP did not change ETCO₂ and heart rate but increased breathing frequency. It implies that conscious participants may change their breathing pattern to adapt to the application of IAP which may also contribute to the increase in the concentration of exhaled protein. However, this was not due to aversive respiratory sensations such as air hunger, unpleasantness and suffocation. This suggests that IAP can be non-invasively applied to conscious humans without aversive effects that would produce avoidance behavior that reduces patient compliance with the method.

The IAP methods applied to conscious human and anesthetized dogs were different in the isolation of the upper airways and mouth. In the conscious human study, the exhaled protein was collected through a filter connected to a mouthpiece. IAP was applied with the subject breathing through the mouth that may wash out protein from the oral cavity in addition to airways and lungs, affecting the protein quantitative results. However, in the anesthetized dog study, IAP was connected to an endotracheal tube that resulted in the square-wave vibration applied only to sublaryngeal airways and lungs which excludes contamination of the filter sample from the oral cavity for the quantitative analysis. Protein concentration was increased in both studies suggesting that the IAP method can increase the amount of protein and other molecules from the lower airways and lung.

The subjects' respiratory sensations were also tested during control and IAP trials. We found that administering IAP did not cause respiratory discomfort in conscious human and two of five subjects even felt they spent less effort breathing in IAP trial. Thus, the IAP method is well tolerated by the conscious subjects and encourages the subjects to continue using the IAP treatment if needed in repeated trials. This is important for patients and especially children to encourage them use the device for diagnosis or monitoring diseases.

The non-invasive IAP of this study increased the amount of exhaled protein without causing respiratory discomfort. Specifically, the use of IAP increases the sensitivity of exhalate monitoring and diagnosis of respiratory infection, inflammation and other pulmonary diseases.

### Example 4

### Materials and Methods

### Experimental Set-Up

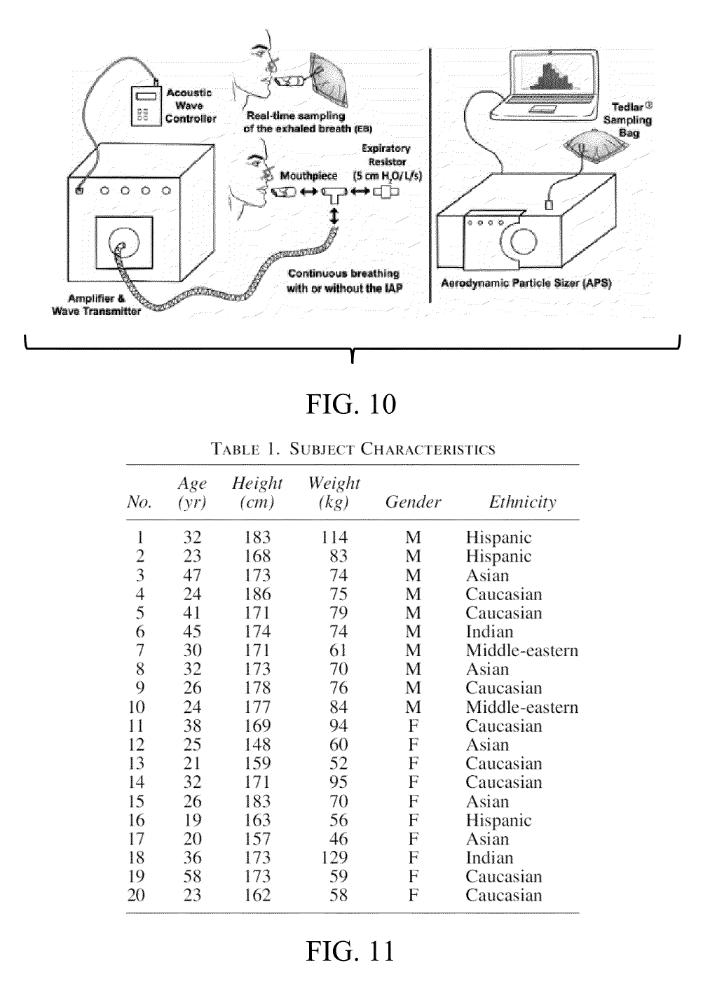

A schematic of the experimental set-up and procedure is presented in FIG. 10. An internal airway percussion apparatus (IAP) was constructed from an acoustic wave controller (HPG1, VELLEMAN® Inc., Fort Worth, Tex.) connected to an amplifier (MG10, Marshall Amplification PLC, Bletchley, Milton Keynes, UK). The IAP delivered acoustic waves to the airways with adjustable waveforms, frequencies, and pressure amplitudes. A pressure transducer (Stoelting 50110, Stoelting Co., Wood Dale, Ill.) was used to measure the pressure amplitude of the IAP and to convert the measured values to the equivalent wave amplitudes. A snorkel-like tube connected to the amplifier was adopted for transmitting the generated waves through a mouthpiece commonly used for pulmonary function tests. To ensure the produced air pressure waves are transmitted down the respiratory system, the opposite end of the mouthpiece was connected to a 5 cm H₂O/L/sec respiratory resistor (Model #7100R, Hans Rudolph, Inc., Shawnee, KS) throughout both the inspiration and expiration portions of the breathing cycle. Sampling bags used for aerosol sampling were 1-liter TEDLAR® bags (SKC Inc., Pittsburgh, Pa.) which had internal dimensions of 241×254 mm² as a deflated bag, with a wall thickness of −50 μm and single polypropylene (hose/valve and septum) fittings.

### Experimental Procedure

The experiment for each subject was composed of a pair of 10-minutes before-and-after trials at a single frequency and amplitude of IAP. All participants were asked to wear a nose clip throughout the trials to ensure optimal delivery of sound waves to, and collection of exhaled breath particles from the lower respiratory system. First, the IAP device was turned off and the participant breathed normally in the sitting position. At the end of every minute, the participant took a deep inspiration, the snorkel-like mouthpiece was removed from the participant's mouth, and immediately exhaled the breath into a sampling bag. After the first 10 minutes, there was a 5-minute break during which the participant removed the nose clip and drank a cup of water. After the break, IAP was switched on to generate the sound waves at the selected frequency and pressure amplitude. The same sampling procedure was repeated when IAP was switched on to compare the results to the baseline with no percussion. Only one trial per participant was performed per day.

Although TEDLAR® bags are well known for their nonabsorptive property, filled sampling bags were immediately analyzed to ensure real-time characterization of the sampled particles. An AERODYNAMIC PARTICLE SIZER® 3321 (APS™ spectrometer, TSI Inc., Shoreview, Minn.), a high-resolution device for real-time measurements of particles with aerodynamic diameters ranging from 0.5 to 20 μm, and AEROSOL INSTRUMENT MANAGER® (AIM) V9.0 software package was used to analyze and record particle size distributions. Particle capture from the sampling bag continued for 60 seconds at a flow rate of 1 L/min. Wilcoxon signed-rank paired test, which is a nonparametric procedure for small populations (first set of experiments), and paired t-test, which is preferred for correlated group design of larger populations (second set of experiments), wherein each subject is tested twice on the same variable, were adopted for inferential statistical analysis of the recorded data for particle sizes and total mass. This was done using SPSS® (V21, IBM Inc., Endicott, NY, USA). The confidence level of 95% was used as the measure for reliability of the results.

The experimental protocol was reviewed and approved by the University of Florida Institutional Review Board. The first set of experiments served as sensitivity analysis on different wave frequencies (15, 30, and 100 Hz) and wave pressure amplitudes (0.75, 1.5, and 3 cm H₂O) on five healthy human subjects (healthy adults: 2 male and 3 female, between the ages of 30 and 40) to identify the most effective combinations. These ranges of pressure amplitude and frequency were adopted based on the literature and tolerable range observed by practices on pulmonary patients. Based on the earlier study described in Example 3 that found a square waveform to be more effective than the other waveforms (sinusoidal, tri-angular or saw tooth), a square waveform was applied in all of the tests.

The second set of experiments focused only on evaluating those frequency/pressure amplitude combinations with a p value about 5%: 15 Hz at 3 cm H₂O and 30 Hz at 0.75 cm H₂O. Twenty nonsmoking healthy adults (10 males and 10 females aged 19-58 years) were recruited for this part of the study (Table 1; FIG. 11).

### Results

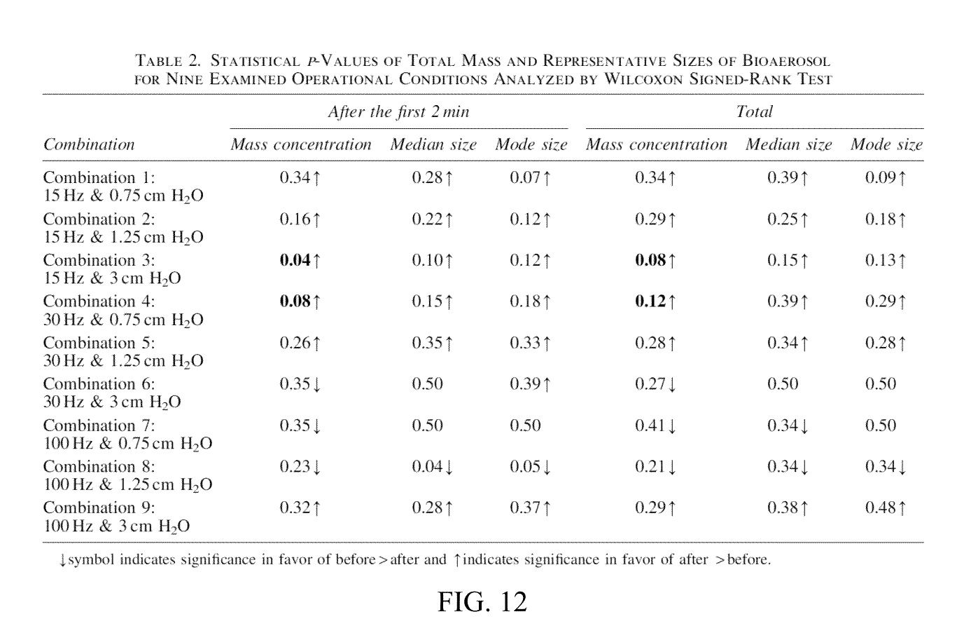

The results of the Wilcoxon signed-rank paired statistical analysis on the first set of experiments after the first 2 minutes and after the entire duration of the experiment are summarized in Table 2 (FIG. 12). As shown, the probabilities of observing an increase in total mass of particles collected from the exhaled breath (EB) for Combinations 3 and 4 after the entire duration of the experiment were 92% and 88%, respectively. Comparing the p values of the first 2 minutes with those obtained after the entire runtime of the experiments, the increase in the total collected mass in the first 2 minutes was the greatest (especially for Combinations 3 and 4). Considering p values of the median and mode sizes of the particles in all combinations, Combination 1 was the only one wherein the mode size of the sampled particles became larger after implementing the IAP device.

According to Table 2, operating the IAP device at wave frequencies above 30 Hz reduced the total mass of the sampled particles. In other words, there was an upper limit for the wave frequency of the IAP device to ensure intensification of the total mass of particles. According to the following Eq. 1:

$$ω \approx \frac{1}{{d}_{d}} \frac{\sqrt{3 {kP}_{0}}}{ρ} \qquad ( Eq . 1 )$$

(where p is the density, P₀ is the static pressure over the frequency of a sound wave travelling from the point of origin (amplifier) to the destination (ALF), k is the wavenumber (ratio of 2 p/wavelength), and d_{d} is the maximum size of an acoustically driven air bubble formed in microtube mixers/transporters), the wave frequency and size of the particle droplets are inversely proportional, justifying the observed threshold (30 Hz). After the first 2 minutes of the sampling, for low or medium pressure amplitudes (0.75 or 1.25 cm H₂O) at the highest wave frequency (100 Hz), the median and mode sizes of the sampled particles also decreased, suggesting that Combinations 7 and 8 are the most ineffective IAP operational conditions. Although further simultaneous increase of the wave frequency and wave amplitude (Combination 9) implied improvement in total mass and particle size, the statistical significance for such conclusion was not close to the 95% confidence level.

Based on the capillary wave theory, the count median diameter (CMD) of the droplets produced through ultrasonic nebulization is inversely related to the wave frequency and amplitude. The experimental correlation of droplet size to wave frequency and wave amplitude in ultrasonic nebulization can be expressed by:

$$CMD = k \frac{4 v}{αω} \qquad ( Eq . 2 )$$

where v is the kinematic viscosity of the fluid and k is a constant ranging from 0.34 to 0.36. The inverse relationship between the median diameter of the particles and wave frequency is consistent with Eq. 1. Increases of both droplet number concentration and droplet mode size are important in intensifying the total sampled particles. However, droplet size enlargement is more influential than the number concentration, as the total particulate mass is proportional to the cube of the particle diameter. As pointed out earlier, increasing both wave amplitude and frequency results in an increase in particle number concentration, but at the same time the average particle size is reduced which complicates prediction for the optimal operational condition of the IAP device.

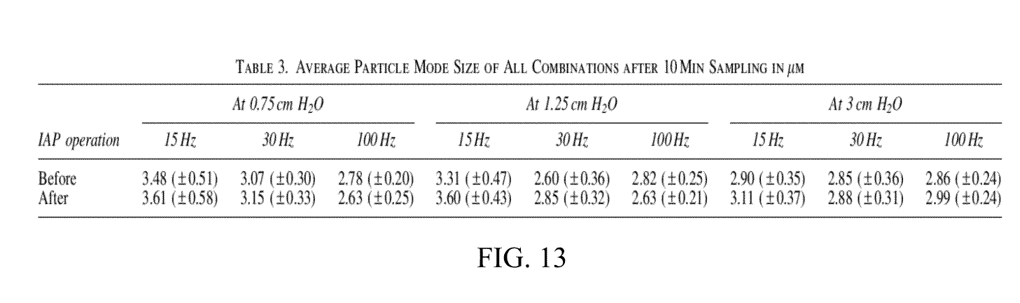

To compare measured particle size to that suggested from the correlation as a function of wave frequency and amplitude (Eq. 2), the median particle sizes of all combinations averaged over all subjects participating in the first set of experiments are summarized in Table 3 (FIG. 13). Consistent with Eqs. 2 and 3, the average particle sizes for before-and-after trials decreased with increasing the wave amplitude and frequency. Meanwhile the higher the frequency, the more stable the particle mode size, as the standard deviation of the results at higher frequencies was relatively lower. Although Combination 1 was the only combination ensuring particle size enlargement with more than 91% confidence, the average increase in its particle mode size was small (˜5%).

Because an increase in total mass of particles in the EB after applying IAP under Combination 1 condition did not reach statistical significance (p-value of 0.34), Combination 1 was not selected for the second set of experiments.

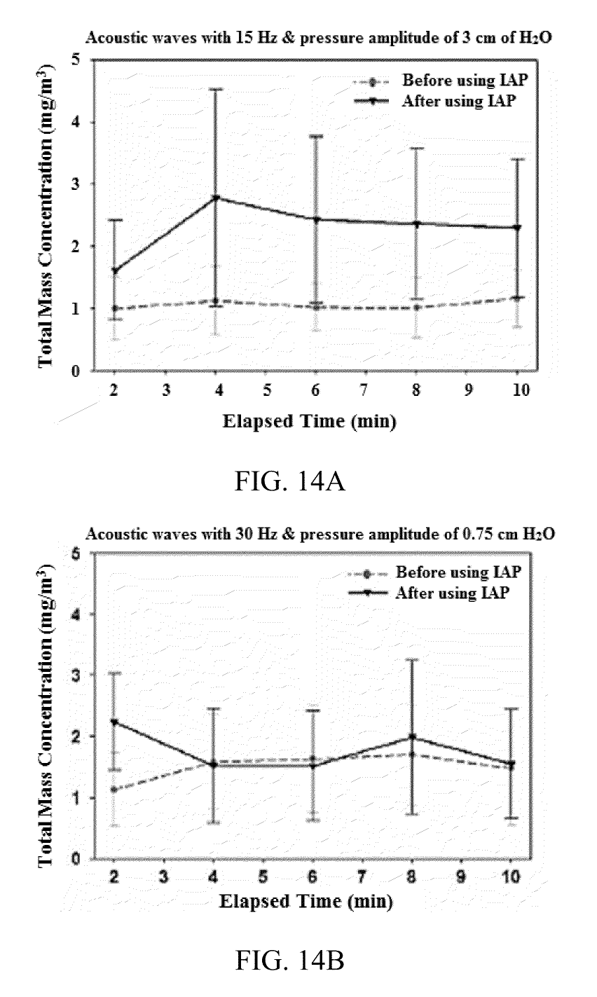

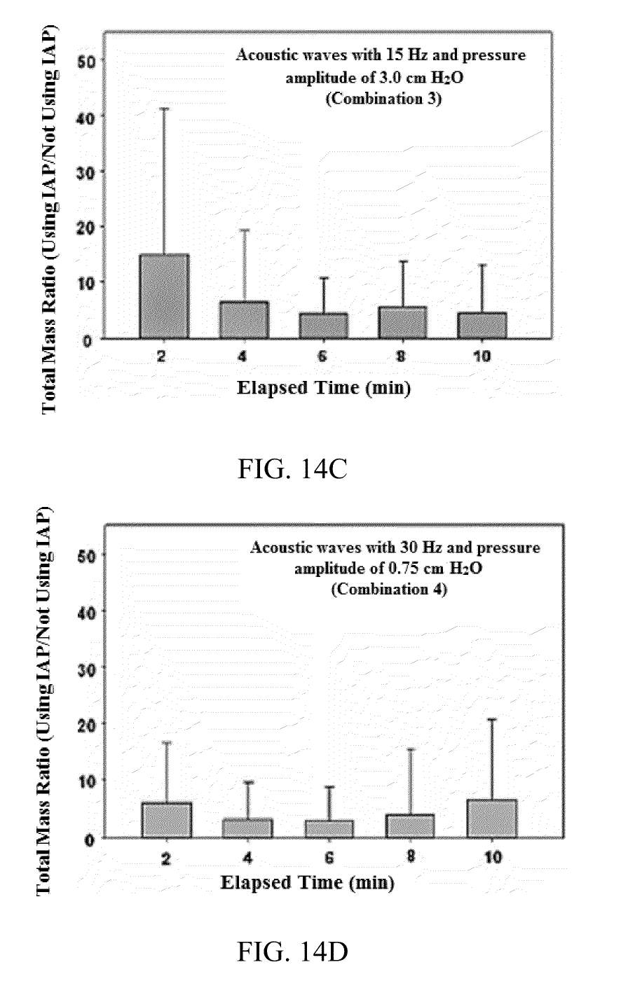

The second set of experiments was conducted with only 2 out of 9 originally evaluated combinations of the IAP device operational conditions in which the total mass of exhaled particles was the greatest (i.e., Combinations 3 and 4). As shown in FIGS. 14A through 14D, the results of this set of experiments on 20 healthy adult participants demonstrated that an IAP device running at 3 cm H₂O pressure amplitude and 15 Hz frequency (Combination 3) was more effective than an IAP device running at 0.75 cm H₂O pressure amplitude and 30 Hz frequency (Combination 4) for bioaerosol sampling from the EB, after each corresponding 2-minute intervals(p-value of 0.05 versus 0.07 for mass of collected particles after only the first 2-minutes of the experiments, and p-value of 0.01 versus 0.17 for total mass of collected particles during the entire run-time of experiment).

As illustrated in FIGS. 14A and 14B, the first couple of minutes of the experiments resulted in an increase in the total mass of collected particles considering all subjects. FIG. 14C shows that the first 2 minutes of sampling had the greatest enhancement of the same subject (mean values of 15 and 6 times higher total sampled mass ratios for Combinations 3 and 4, respectively). This means that although the average increase in total mass of collected particles was higher after 4 minutes in Combination 3, the percentage of such an increase was not as high as in the first 2 minutes. Similarly, the absolute value of the total sampled mass and enhancement ratio for each subject was initially the greatest in Combination 4. The error bars in FIGS. 14A-14D bound 95% confidence level of the results. This implies application of IAP in the first 2-minutes of the trial may be sufficient to increase the particle collection, since during the first 2 minutes most particles were initially exhaled leaving fewer particles to be exhaled in the latter minutes.

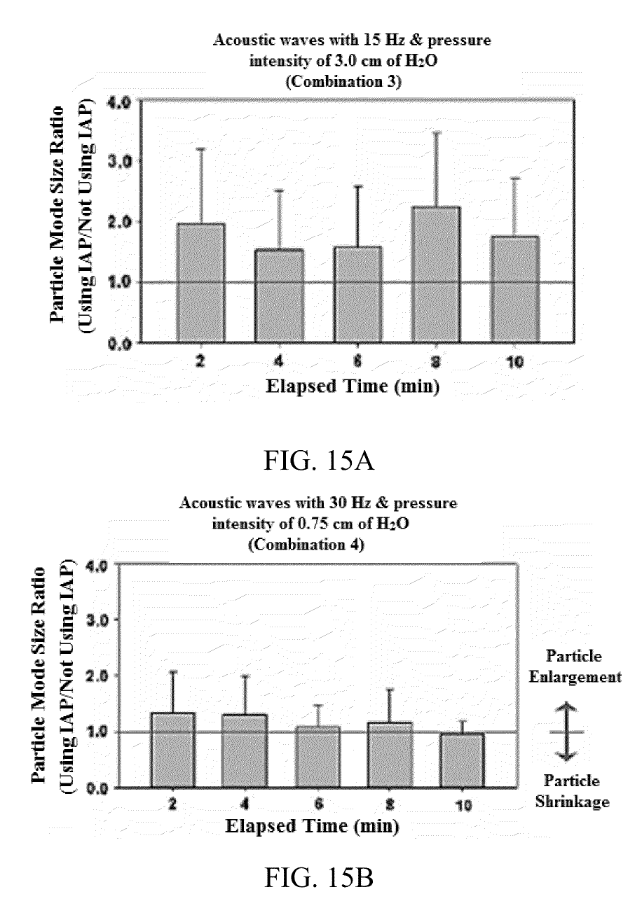

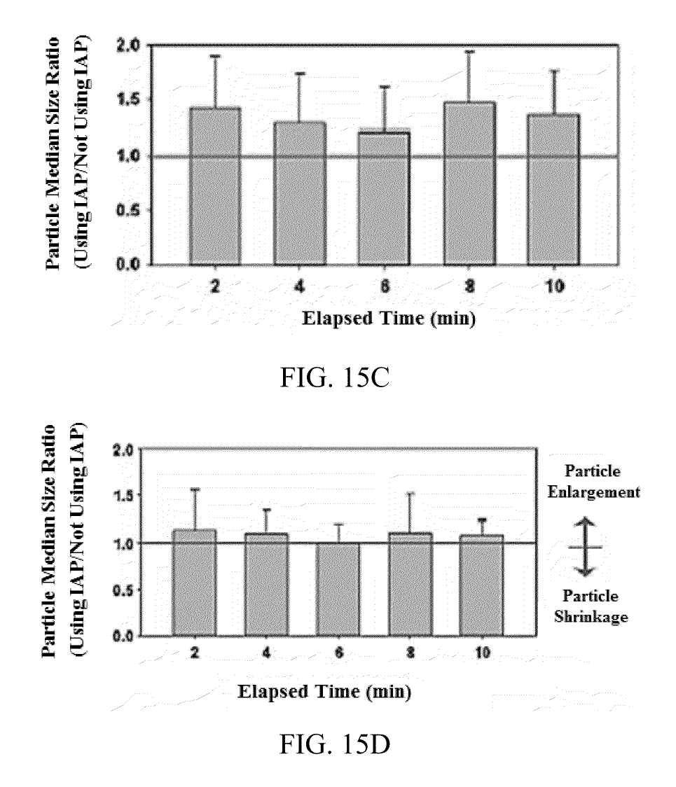

As presented in FIGS. 15A and 15C, there was a 1.25-2.25 times increase in the average mode diameter (increasing from 3.2, 3.2, 3.6, 3.0, and 4.0 μm to 4.8, 4.0, 4.4, 6.0, and 5.1 μm, respectively) and a 1.2-1.5 times increase (increasing from 3.0, 3.10, 3.6, 2.9, and 3.8 μm to 4.2, 3.9, 4.3, 4.2, and 4.9 μm, respectively) in the average CMD of the sampled particles for Combination 3. On the other hand, a smaller increase (average 10% enlargement) was observed for Combination 4, mostly within the first 4 minutes of the experiment. Gradual drying out of the airways due to implementation of a higher frequency compared to Combination 3 may be responsible for the observed drop in the particle size ratio.

The experimental results showed that predicting the CMD of the sampled particles using Eq. 2, which is a correlation for ultrasonically nebulized particles, is not the best fit for TAP operation. The reason is that Combination 3 with two times greater wave intensity compared to Combination 4, concluded higher CMD values. However, obtained results are consistent with Eq. 1, suggesting inverse relationship between the wave frequency and the size of the droplets. This also means doubling the wave intensity (product of the wave frequency and amplitude) in Combination 3 from that of Combination 4 is the most important factor.

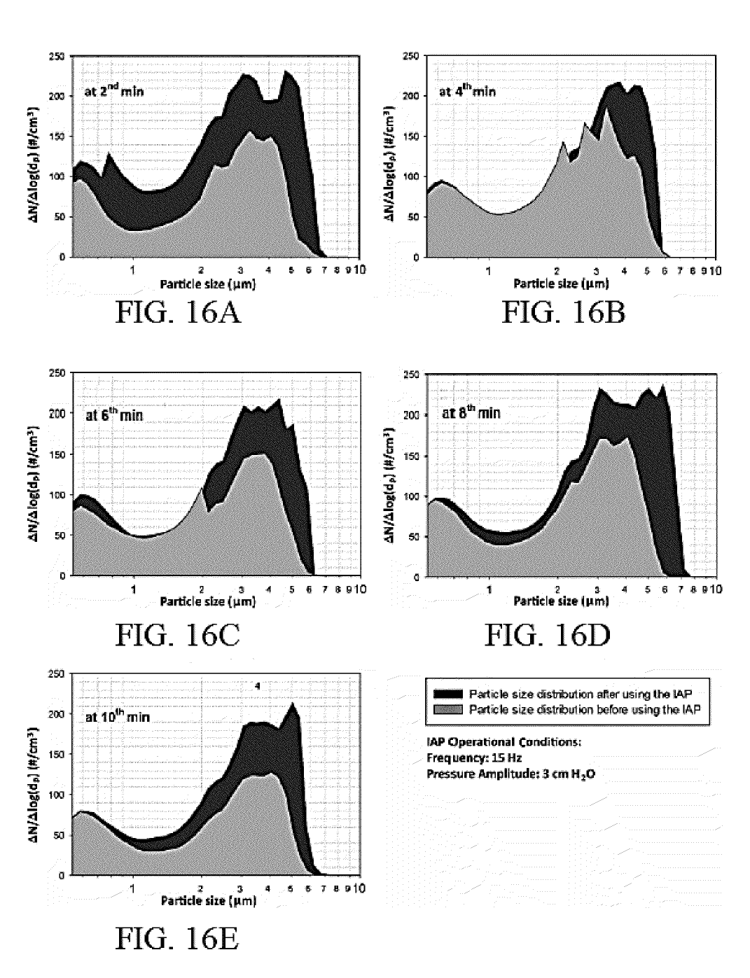

To date, no previous study has investigated the particle size distribution (PSD) of the EB particles using an APS spectrometer for particles between 0.5 and 20 μm during normal breathing of healthy adults. The PSD plots of the collected particles at different sampling times for Combination 3 measured by APS spectrometer and processed by AIM® V9.0 are presented in FIG. 16.

FIG. 16 confirms that the first 2 minutes of the experiment had the highest intensifying impact on the sampling particles, as the number concentration of particles in the entire size range was greater after TAP application. Since the number concentration of particles in all sampling minutes of each plot was approximately the same, sound waves with relatively lower frequency of Combination 3 did not dry out the ALF within 10 minutes.

According to FIG. 16, application of the TAP with operational conditions of Combination 3 increased the range of particle mode sizes from (2.5-3.5 μm) to (3.5-7 μm). The mode values were smaller than 8.53 μm, which was the previously reported mode size for exhaled particles during coughing (see Yang et al., “The size and concentration of droplets generated by coughing in human subjects.” J Aerosol Med. 2007; 20:484-494). Likewise, the entire range of size distribution (0.5-8.6 μm) in this study was narrower than the entire range of size distribution during coughing (0.5-15.9 μm) studied by Yang et al.

Although the APS Spectrometer is capable of detecting particles with aerodynamic diameter up to 20 μm, no particle larger than 8 μm was recorded in all plots. A sharp drop in particle concentration at around 8 μm is observed that may be due to the loss of particles larger than 8 μm. Terminal settling velocity of these particles (>8 μm) is sufficiently high (e.g., ˜0.3 cm/sec for 10-μm particles at STP), allowing them to settle on the inner walls of the sampling bag or tubing (averagely 2.5 cm away from the centerline of the air flow) during their travel between the sampling bag and the APS inlet (60 sec at 1 Lpm for full evacuation of the 1-liter sampling bag). Moreover, previous results from a study revealed some liquid particles deposits at the beveled tip of the inner nozzle at the APS entrance. This trend intensifies with an increase in particle size, and for droplets larger than 10 μm, the fraction of detected droplets becomes as low as 25%. This may account for not detecting droplets larger than 8 μm in all trials.

The IAP device, in contrast to flutter and acapella devices, is not dependent on the performance of a specific breathing task and ergonomic factors such as the breathing flow rate and effort. No discomfort was felt by subjects of the study, suggesting that IAP respiratory effort is appropriate for children and patients with difficulty in controlling their breathing. The HFO performance was only tested on the marked 5-μm polystyrene particles deposited in the lungs for mechanical ventilation but not sampling polydisperse bioparticles from the EB. Since the HFO device has never been studied for bioaerosol sampling from the exhaled breath, direct comparison of the IAP device to the HFO performance reported in the literature should be cautioned. Nevertheless, the IAP device operated at its optimal operational conditions can be expected to be more efficient than the HFO device for increasing the mass of aerosolized particles based on the results of 5-μm particles in that study. Similarly, improved effectiveness of the IAP in clearing particles/mucus from the human respiratory system is anticipated for patients with pulmonary infections.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

We claim:

1. A method of clearing mucus from airways of a patient using a device for applying an oscillating airflow to the air passing through the airways of the patient, wherein the device comprises,
a. a mouthpiece, and
b. an oscillator operably connected to the mouthpiece,
and collecting a sample from the patient following application of oscillating airflow to the patient;
wherein the device creates turbulence throughout the airways of the patient from the mouth to the alveoli when the patient breathes through the mouthpiece, and
wherein the device comprises a speaker or a piston pump.

2. The method of claim 1, wherein the patient has cystic fibrosis, bronchiectasis or other chronic lung diseases associated with mucus hypersecretion.

3. The method of claim 1, wherein the patient is a pediatric patient.

4. The method of claim 1, wherein the device is portable.

5. The method of claim 1, wherein the device is battery powered.

6. The method of claim 1, wherein the device comprises a speaker connected to a frequency generator and amplifier.

7. The method of claim 1, wherein acoustic sound waves are applied with the device.

8. The method of claim 7, wherein the acoustic sound waves are selected from the following frequencies: 15 Hz, 30 Hz, 60 Hz, and 100 Hz.

9. The method of claim 7, wherein the acoustic sound waves are applied at the following intensities: 0.75 cm H₂O, 1.5 cm H₂O and 3 cm H₂O.

10. A device for applying an oscillating airflow to airways of a patient, the device comprising:
a. a mouthpiece, and
b. an oscillator operably connected to the mouthpiece,
wherein the oscillator creates turbulence throughout the airway from the mouth to the alveoli when the patient breathes through the mouthpiece, and
wherein the oscillator is a speaker or a piston pump.

11. The device of claim 10, wherein the oscillator is a speaker that transmits square form audio waves to the airways of the patient through the mouthpiece.

12. The device of claim 11, wherein the speaker is connected to a frequency generator and amplifier.

13. The device of claim 10, wherein the device is portable.

14. The device of claim 10, wherein the device is battery powered.

15. The device of claim 10, wherein the oscillator is connected to the mouthpiece via a side port.
